(19) United States
(12) Patent Application Publication
(10) Pub. No.: US 2024/0299649 A1
(43) Pub. Date: Sep. 12, 2024

(54) SENSING AND CONTROL SYSTEM FOR AN IMPLANTABLE INFUSION PUMP

(71) Applicant: CraniUS LLC, Baltimore, MD (US)

(72) Inventors: Owen FRIESEN, Baltimore, MD (US); John CAI, Baltimore, MD (US); Nathan SCOTT, Baltimore, MD (US); Charles WATKINS, Baltimore, MD (US); Anthony GARCIA, Baltimore, MD (US); Conner DELAHANTY, Baltimore, MD (US); Farooq AKHTAR, Baltimore, MD (US); Rahul GANGWANI, Baltimore, MD (US); Eleni DASKOPOULOU, Baltimore, MD (US)

(73) Assignee: CraniUS LLC, Baltimore, MD (US)

(21) Appl. No.: 18/599,594

(22) Filed: Mar. 8, 2024

Related U.S. Application Data
(60) Provisional application No. 63/489,493, filed on Mar. 10, 2023.

(51) Int. Cl. A61M 5/142

(52) U.S. Cl. CPC A61M 5/14276; A61M 2005/14208; A61M 2205/0227; A61M 2205/0233; A61M 2205/04; A61M 2205/106; A61M 2205/3313; A61M 2205/3327; A61M 2205/3334; A61M 2205/50; A61M 2205/8206

(57) ABSTRACT

According to at least one exemplary embodiment, an implanted medical pump sensing and control system may be provided. The pump sensing and control system may include a pump management circuit board. The pump sensing and control system may further include an implanted mainboard within a device, which may be a multilayer PCB that houses the control, communication, and power systems for the entire implanted device. The implanted mainboard may include one or more of a processor, a voltage control circuit, and/or a pump control circuit. The implanted mainboard may further communicate with the pump management board, and thereby control either a unidirectional or bidirectional pump.

### BACKGROUND

Implanted drug delivery devices reduce required and/or repetitive surgeries, target specific areas of the body to increase drug safety and efficacy, and ease the process of providing lifesaving medicine. This is particularly advantageous when surgery is deemed suboptimal, for example for chronic disease management in instances when critical organs within the human body cannot be resected or excised. A key function of these devices is the ability to precisely control and monitor how much infusate is introduced to the target body part and/or organ. As a result, there is a critical need to ensure that implanted drug delivery devices deliver a consistent dosage which avoids over/under delivery of infusate. As such, the infusion pumps, and their sensing and control systems contained within these implanted devices play an important role in reliably delivering medicine.

Since the implanted components are within the human body, including potentially inside the cranium and near the brain, there are additional considerations beyond conventional electromechanical systems. These include constraints on size, flexibility, precise microdosing with narrow therapeutic windows, and power consumption.

### SUMMARY

According to at least one exemplary embodiment, a medical implant pump sensing and control system may be provided. The pump sensing and control system may include a pump management circuit board. The pump sensing and control system may further include an implanted mainboard within a device, which may be a multilayer printed circuit board (PCB) that houses the control, communication, and power systems for the entire device. The implanted mainboard may include one or more of a processor, a voltage control circuit, and/or a pump control circuit. The device's mainboard may further communicate with the pump management board, and thereby control a pump.

### BRIEF DESCRIPTION OF THE FIGURES

Advantages of embodiments of the present invention will be apparent from the following detailed description of the exemplary embodiments. The following detailed description should be considered in conjunction with the accompanying figures in which:

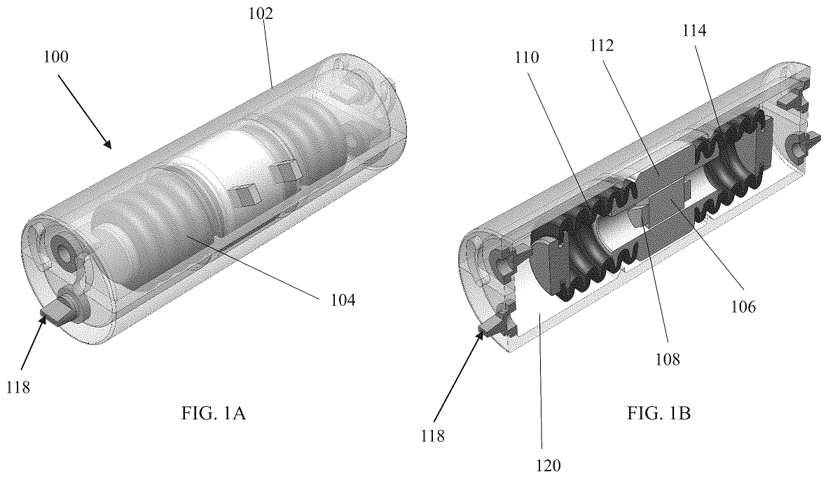

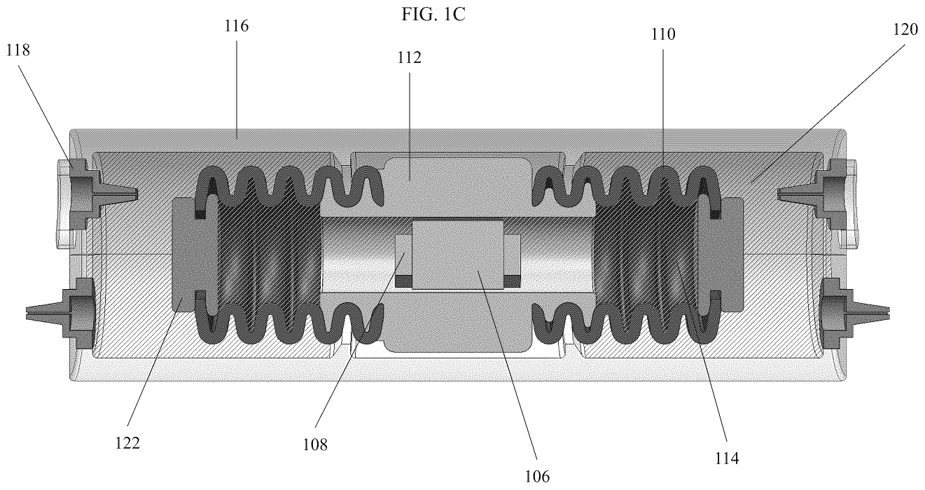

Exemplary FIG. 1A shows an embodiment of an electroosmotic pump device with an external housing surrounding an inner assembly.

Exemplary FIG. 1B shows an embodiment of the cross-sectional view of the device inner assembly with elements of the inner assembly shown.

Exemplary FIG. 1C shows an embodiment showing cross-section of the electroosmotic pump device, with internal elements of the pump shown.

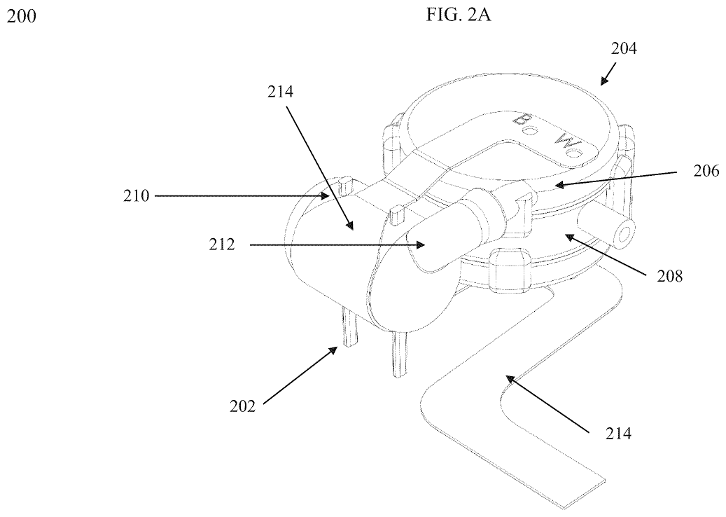

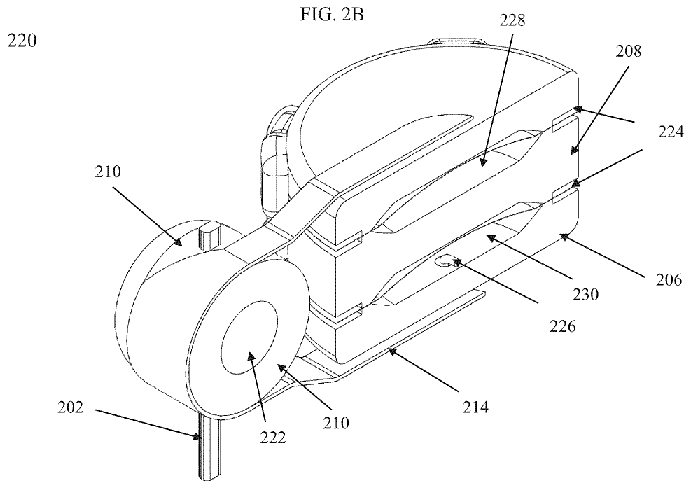

Exemplary FIG. 2A shows an exemplary alternative embodiment of an electroosmotic pump.

Exemplary FIG. 2B shows a cross-sectional view of the exemplary alternative embodiment of the electroosmotic pump.

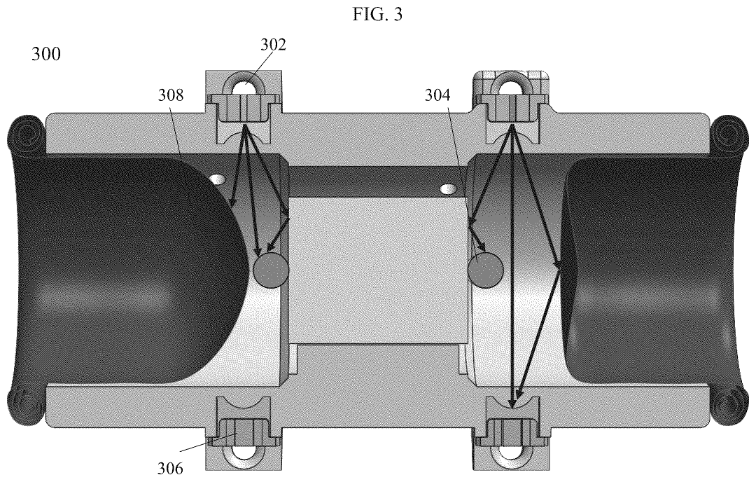

Exemplary FIG. 3 shows an exemplary embodiment of a cross-section of a phototransistor sensing mechanism.

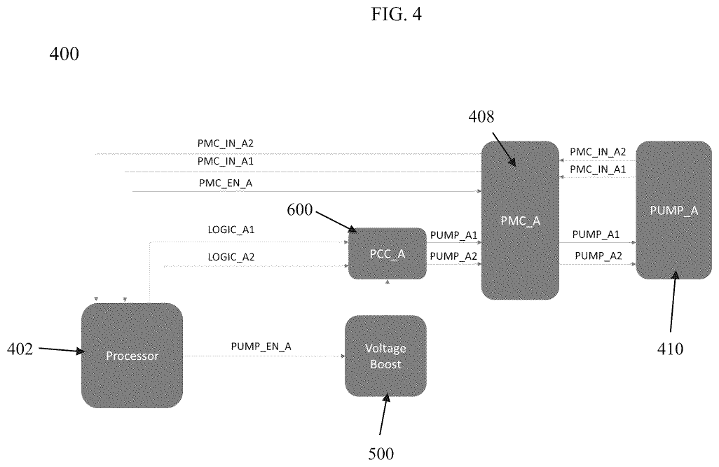

Exemplary FIG. 4 shows an exemplary logic interface between components of the pump sensing and control system and the pump.

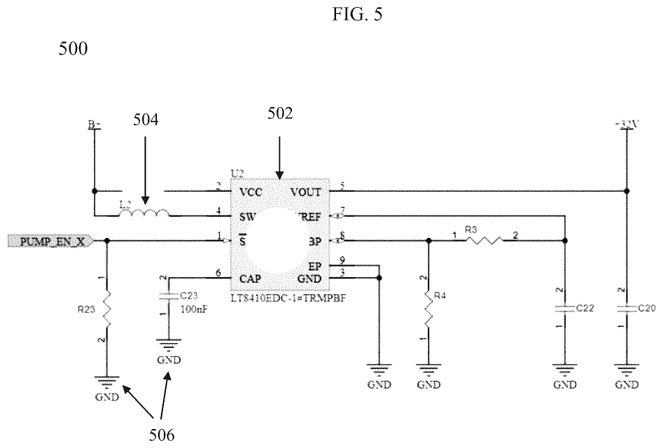

Exemplary FIG. 5 shows an exemplary voltage control circuit.

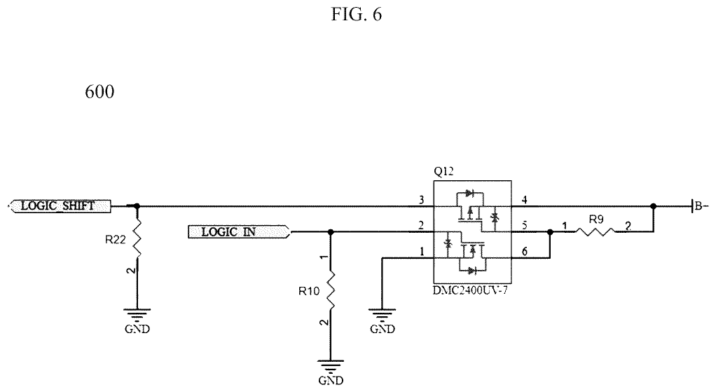

Exemplary FIG. 6 shows an exemplary pump control circuit.

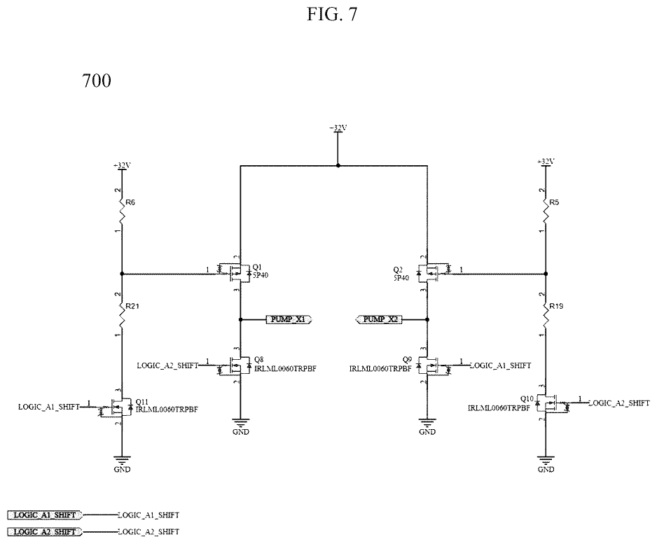

Exemplary FIG. 7 shows an exemplary h-bridge.

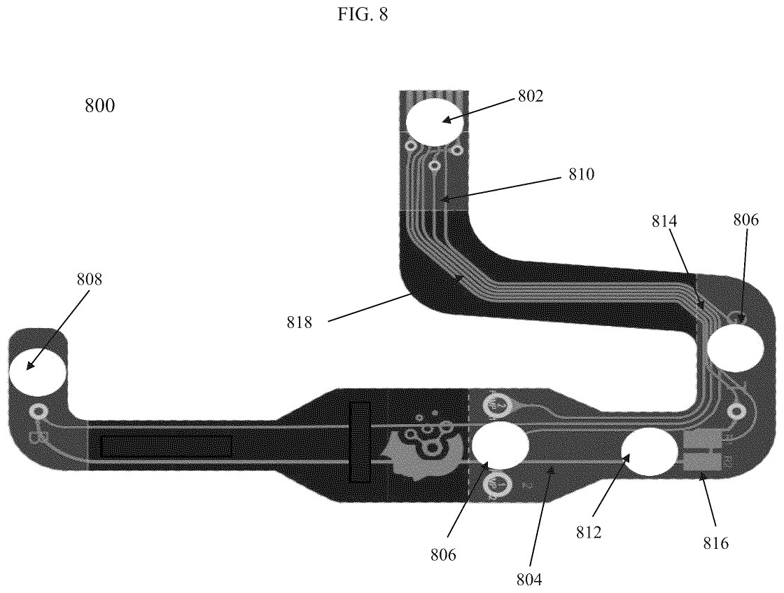

Exemplary FIG. 8 shows an exemplary pump management board.

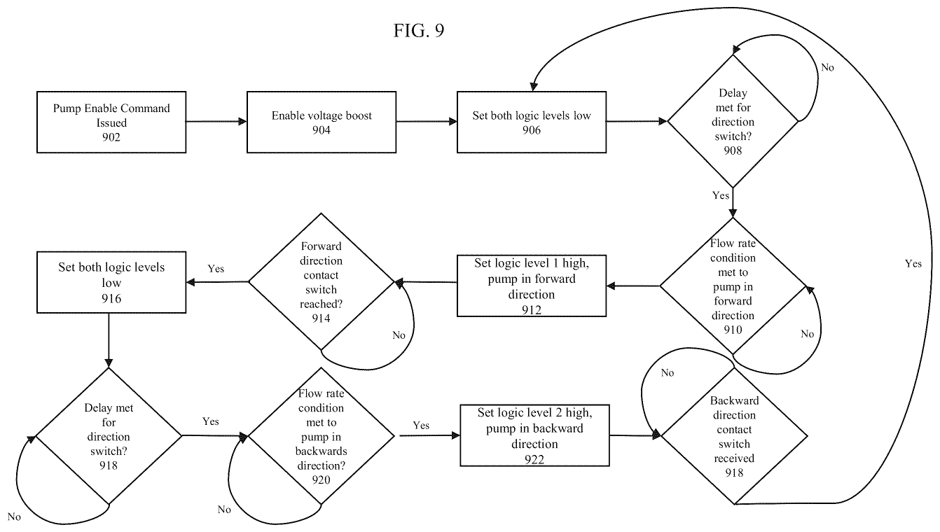

Exemplary FIG. 9 shows an exemplary pump control system logic diagram.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the invention”, “embodiments” or “invention” do not require that all embodiments of the invention include the discussed feature, advantage or mode of operation.

As used herein, electroosmotic element (EOE) means a structure that, when a voltage is applied, moves a fluid towards an electrode. By alternating the polarity of the applied voltage, the EOE can create reciprocating fluid motion, functioning as a bidirectional electroosmotic pump. Advantages of being bidirectional may include a smaller footprint for controlling flow through two different catheters as opposed to just one, which may be valuable in areas such of the cranium.

As used herein, electroosmotic pump (EOP) means a structure that is capable of delivering a fluid, at a precise flow rate, through the use of an EOE.

As used herein, it may be understood that MRI-safe means the device, when used in the MRI environment, presents no additional risk to the patient or other individual, but it may affect the quality of the diagnostic information.

As used herein, it may be understood that MRI-compatible means the device, a device that is MRI safe when used in the MRI environment and does not significantly affect the quality of diagnostic information taken by the MRI or have its operations affected by the MRI system.

FIG. 1A depicts an embodiment of an electroosmotic pump device 100 with an external housing 102 surrounding an inner assembly 104. It may be appreciated that the internal elements may be formed out of any of a variety of materials or combinations thereof, based on implementation and use. Further, in the embodiments, the components utilized in the pump assembly may be biocompatible with the human body and may be absent of any ferrous-containing elements, which can mitigate adverse effects related to MRI equipment or imaging conditions. Thus, in some further embodiments, the embodiments may include a pump or pumps that are MRI-safe, MRI-compatible, and/or MRI-lucent (i.e., does not obscure target sites on imaging and thereby able to prevent suboptimal imaging value for pathology assessment). In some embodiments the pump or pumps may be bidirectional pumps. The external housing 102 may be cylindrical or, in other embodiments, a different shape. Therefore, the housing 102 may be formed and utilized in such a manner as to minimize space needed for human body implantation. The external housing 102 of the device may be formed from a biocompatible and durable plastic or photocured resin, for example any alloplastic material approved for human use by the FDA. However, other shapes and/or materials may be used based on the use and implementation of the pump for certain disease conditions and anatomical variance (i.e. placement within head, chest, abdomen, or an extremity, as desired), and compatibility of the other materials in the pump, including, for example, the working fluid and payload fluid. Each end of the external housing 102 may include one or more valves 118. The one or more valves 118 may be, for example, duckbill valves, and may be formed from silicone or another MRI-lucent material to facilitate the flow of a payload fluid 120. In an exemplary embodiment, the valves 118 may be poppet valves. In other embodiments diaphragm valves or duckbill valves may be used. Further, duckbill valves may be easily accessible and easily integrated into the pump. Other types of valve systems may include, but are not limited to, custom valve flaps, umbrella valves, or other flow metering systems, as desired. In an exemplary embodiment, there may be four valves in combination, two of which facilitate the unidirectional flow of fluid into the pump and two of which facilitate driving unidirectional fluid out of the pump. Of note, unidirectional flow may protect the medicine from flowing back into the pump, which would cause the target organ or body part to receive less of the necessary medicine or therapy. In other embodiments there may be fewer valves utilized, which may be accomplished, for example, by combining fluid channels so there is one inlet and one outlet valve. However, it may be understood that in other embodiments, only two valves may be used, or more than four valves may be used, depending on application.

FIG. 1B depicts an embodiment of the cross-sectional view of the device inner assembly 104 with elements of the inner assembly shown. In an embodiment, the inner assembly 104 may contain the electroosmotic element 106 which may be contained by a holder 112 and may receive a voltage from electrodes 108. These electrodes may be platinum or palladium, with the latter having an inherent advantage of resorbing unwanted bubbles secondary to hydrolysis. The holder 112 can be connected to bellows or diaphragms 110 which can contain a working fluid 114. External to, or surrounding, the inner assembly 104 is the payload fluid 120 which is contained by the external housing 102. It should be understood that this is merely an exemplary configuration and, depending on application or location of an implant, different configurations may be used. In addition, the bellows or diaphragms can be shaped and designed with various forms to improve long-term conditions, enhance function, and/or minimize wear, tear, or degradation. Titanium diaphragms, as opposed to rubber bellows, may have a longer lifespan which would equate to improved safety and efficacy.

FIG. 1C depicts an embodiment showing a cross-section of the electroosmotic pump device 100, with internal elements of the pump shown. It may be appreciated that the internal elements may be formed out of any of a variety of materials or combinations thereof, based on implementation and use. In an embodiment, the electroosmotic device 100 may include an electroosmotic element 106 that may be formed by, for example, using a ceramic cylinder contained by a holder 112, which may be formed, for example, with polyphenylene sulfide, polyethylene terephthalate, glass, titanium, high-density polyethylene (HDPE), polycarbonate, or photocured resin. In an exemplary embodiment, incorporating a ceramic element may be done to avoid any ferrous-containing materials. This may allow the pump(s) and/or any implants using the pump(s) to increase patient safety and enhance pump functionality, for example with respect to MRI imaging conditions. Depending on application, in some embodiments ferrous-containing elements may not only jeopardize safe and reliable pump function post-MRI, it may also risk potential dislodging and/or life-threatening extrusion of the pump when exposed to magnetic fields.

Still referring to FIG. 1C, on either side of the electroosmotic pump 100 may be electrodes 108, which may be formed of platinum, palladium, or another material capable of transferring electricity. Platinum, in an embodiment, may be utilized for its attractive material properties with respect to size, length, and flexibility. In an embodiment, platinum may be utilized because it can interact with various working fluids while remaining inert. Further, the platinum may be formed into a desirable shape while maintaining electrical contact with the electroosmotic element 106. In another embodiment the electrodes 108 may be formed from palladium which may limit gas bubble generation. Notably, other materials may develop an oxidation layer when an electrical charge is applied. The electrodes 108 may create an electric field through the electroosmotic element 106 that can cause pumping actuation to occur, for example in alternating directions (i.e. bidirectional). The electroosmotic element holder 112 may be connected to each bellows or diaphragms 110, which may be positioned on either side of the holder 112, and which may be capped by plugs 122. In some embodiments the bellows or diaphragms 110 may form an uninterrupted part and no plug 122 may be needed. The bellows or diaphragms 110 may be formed from titanium, silicone, fluoroelastomers, or other materials such as latex and the plugs 122 may be formed from photocured resin, as desired. The bellows or diaphragms 110 shape in the embodiments may be such that it may minimize size, maximize function, prevent cracking, and/or reduce long-term wear or degradation associated with high-volume and frequent movements required in use in some embodiments. The plug can be made from photocured resin, silicone, or other polymers, all of which may reliably bind to the bellows or diaphragms 110. The plug can also be drilled so as to allow for filling with the working fluid 114, and then may be resealed with additional photocured resin. The bellows or diaphragms 110 may contain a working fluid 114 which may facilitate the deformation of the bellows or diaphragms 110. This working fluid, in an exemplary embodiment, may be strategically chosen based on its safety profile, efficiency in conducting electricity, and/or its stability over time with respect to bubble formation, and may be, but are not limited to, an aqueous buffered solution, ethanol, or dimethyl sulfoxide (DMSO). Surrounding the holder 112 and the bellows or diaphragms 110 may be a casing 116 (for example formed of photocured resin) that can contain an inner assembly 104 and the payload fluid 120. On each end of the casing 116 there may be two valves 118, for example formed of silicone, which can facilitate flow of the payload fluid 120 in and out of the electroosmotic device 100.

FIG. 2A depicts an exemplary alternative embodiment of an electroosmotic pump. The exemplary EOP 200 design may have one or more electrodes 202 that may power the EOP 200. The one or more electrodes 202 may be made out of, for example, platinum or palladium. The EOP 200 may further have an EOP bellow housing 204 which may further have an outer housing 206 and an inner housing 208. The bellow housing 204, outer housing 206, and inner housing 208 may be made of, for example, titanium, another metal, Polyphenylene Sulfide (PPS), plastics, polymers, and/or any other materials known in the art. In some embodiments the outer housing 206 and the inner housing 208 may be made of the same material while in others they may be made of different material. The inner housing 208 and outer housing 206 may be connected through, for example, welding, epoxy, or ultrasonic welding. The EOP 200 may further have an electroosmotic element housing 210. The EOE housing 210 may be connected to the bellows or diaphragms housing 204 by a connector 212. The EOE housing 210 may be made of, for example, polymers, titanium, another metal, glass frit and/or ceramics. A working fluid may further be contained in the EOE housing 210. It may be understood that the working fluid may be able to move between the EOE housing 210 and the EOP bellows or diaphragms housing 204 via the connector 212. The EOP 200 may further be connected to a pump management PCB 214 which may be connected to and control the electrodes 202 and/or to bellows or diaphragms sensing wires 226.

FIG. 2B depicts a cross-sectional view 220 of the exemplary alternative embodiment of the electroosmotic pump 200. Within the electroosmotic element housing 210 there may be an electroosmotic element 222. Within the electroosmotic element housing 210 there may be an electroosmotic element 222. The electroosmotic element 222 may be a material such as ceramic or glass, that when voltage is applied to the electroosmotic element 222 through the electrodes 202 the working fluid is moved. When the voltage being applied to the electrodes 202 is alternated between a first polarity and a second opposite polarity a reciprocating fluid motion may be generated through the movement of the working fluid. This movement may help control movement of bellows or diaphragms 224, for example by the movement of the working fluid via the connector 210 into one or more working fluid chambers 228. The bellows or diaphragms 224 may be, for example, a titanium foil or PTFE sheet that is formed into a dome shape and deforms under pressure. In an exemplary embodiment the bellows or diaphragms may be, for example, 10-microns thick, and may be formed by hydroforming. The bellows or diaphragms 224 may be connected to the EOP bellows or diaphragms housing via, for example, welding. The bellows or diaphragms 224 may further be connected to the PCB via a platinum, palladium, or other material, wire. The movement of the bellows or diaphragms 224 may be detected and recorded by one or more contact sensing electrodes 226 which may be contained within one or more of the working fluid chambers 228 and/or payload fluid chambers 230. The bellows or diaphragms contact sensing electrodes 226 may be attached using, for example, epoxy. The contact sensing electrodes 226 may work by, for example, sensing an electrical connection created when the bellows or diaphragms 224 deform and come into contact with the contact sensing electrodes 226. When the contact sensing electrodes 226 sense such a connection or otherwise determine that the bellows or diaphragms have been deformed, the electrodes 202 may switch polarity, which may begin a new pumping cycle.

Referring generally to FIGS. 1a-2, the sensing system may now be described in more detail. In an exemplary embodiment a PCB or other electrical system may operate as a pump management board. The one or more bellows or diaphragm sensors may be further connected to and controlled by the pump management board. For example, in an exemplary embodiment, the pump management board may electrically pull an electrode of the bellows or diaphragms sensor to a system high (for example 1.8V) at a predetermined interval, for example every second. In other embodiments the system high voltage and the predetermined interval may be different. When the bellows or diaphragm comes into contact with the bellows or diaphragm sensor the voltage of the bellows or diaphragm may pull the electrode down to system ground, which may be registered by the system as a contact event and therefore indicate to the system that the pump has finished pumping in that direction (depending on whether the triggered sensing electrode was in the working fluid chamber or the payload fluid chamber). The pump control system may further apply a voltage across the one or more electrodes which may drive the working fluid in order to deform the membrane. It may be understood that in some embodiments the voltage may be fixed, while in other embodiments the voltage may be modulated by the pump control system based on one or more factors, for example based on feedback from the bellows or diaphragms switches, sensors, or other inputs. System design may be adjusted to maximize the lifespan of each pump system, and inadequate timing may result in premature pump failure. In an exemplary embodiment the pump control system may use pulse width modulation to adjust the effective voltage on the electrodes thereby adjusting the pump flow rate. This may be triggered by, for example, a control loop such as a proportional integral derivative controller, which may adjust the flow rate to maintain a desired pressure at an outlet catheter. In some embodiments other sensors may be used instead of or in addition to the control loop, for example a temperature sensor or another sensor that measures values that may affect the flow rate.

In an alternative embodiment, a phototransistor may provide an alternative method of contact sensing for a pump sensing mechanism. The phototransistor may use IR LEDs and phototransistors to sense the state of a membrane separating the working and payload fluids. An alternative shape may be utilized, for example a shape consisting of a dome made from silicone or other elastic material. The pump may be able to utilize a LED-phototransistor combination to detect how deformed or compressed the rubber dome is at any point in time and detect the pumps pumping cycle. For example, if the elastic dome is extended towards the EOE, the phototransistor may read less LED IR signal and vice versa, when the dome is completely collapsed, the phototransistor may read more LED IR signal.

FIG. 3 depicts an exemplary embodiment of a cross-section of a phototransistor sensing mechanism 300. The phototransistor sensing mechanism 300 may have an IR LED 302 which transmits light across the diameter of the pump assembly. Further, there may be a platinum or palladium wire 304 which provides a barrier for the light to pass through to fully close the light sensing circuit. The phototransistor sensing mechanism 300 may also have an IR phototransistor 306 that detects deformation of a rubber dome 308. The rubber dome 308 may be dipped in another material, for example graphene, on the internal and/or external faces. In other embodiments the dome 308 may instead be made of fluorinated carbon-based synthetic rubber (FKM), Fluorosilicone, PTFE, PMMA, PEEK, or any other similar material. The rubber dome 308 may be able to be deformed at the tip and collapse into itself. This motion may allow for an optical path between the LED 302 and the phototransistor 306. By determining whether the path exists or is blocked the state of the bellows or diaphragms may be determined.

In other embodiments, as an alternative to physical bellows or diaphragms to separate working and payload fluid, a fluid membrane may be used. The fluid membrane could be, for example, a microfluidic channel, a liquid metal, oil, or any other fluid that will not be absorbed into the working or payload fluid. This fluid membrane may achieve the same interaction as a solid barrier provided by a bellows, diaphragm, or rubber dome. The fluid membrane may be moved, therefore moving the payload fluid, by, for example, being oscillated by the active electroosmotic element (EOE) and applied voltages.

In an exemplary embodiment the components may be bonded together using a one-part, biocompatible, room temperature vulcanizing (RTV) silicone.

In an exemplary embodiment the electroosmotic pump(s) may be integrated into an implanted medical device case. This may allow for outlet pathways to exit ports for the payload fluid to also be embedded into the implanted medical device case. The pump(s) may be connected by, for example, manifolds, silicone tubing, and/or fitting pieces.

Discussing now the pump sensing and control system the pump sensing and control system may include the pump management circuit board shown above. The pump sensing and control system may further include an implant mainboard, which may be a multilayer PCB that houses the control, communication, and power systems for the entire implant. In some embodiments the pump sensing and control system may include a plurality of boards each of which may include one or more control, communication, and/or power systems. The implant mainboard may include one or more of a processor 402, a voltage control circuit 500, and/or a pump control circuit 600. The implant mainboard may further communicate with the pump management board 408, and thereby control a pump 410.

FIG. 4 may depict an exemplary logic interface between components of the pump sensing and control system and the pump 400. The processor 402 may have a plurality of pins that interface with the other parts of the pump system. For example, a Pump_en pin may enable a first pump when driven high and may disable the first pump when driven low. It may be understood that the signal being read may be a digital or analog signal depending on the particular application. For example, in a phototransistor sensing system embodiment PMC_IN_A2 & PMC_IN_A1 may be analog signals, while in a bellows or diaphragms sensing embodiment PMC_IN_A2 & PMC_IN_A1 may be digital signals. In an exemplary embodiment one or more logic pins, such as logic_A1 and/or logic_A2 may inform which direction the pump is pumping, for example when A1 is high it pumps in a first direction and when A2 is high it pumps in a second direction. It may be understood that in some embodiments both A1 and A2 may need to first both be set to low before the pump starts or changes directions. In some embodiments there may be a time requirement for how long A1 and A2 have been low, for example 1 sec, 5 sec, etc.

FIG. 5 may depict an exemplary voltage control circuit 500. The exemplary voltage control circuit may increase the voltage provided by the battery onboard the implant to a voltage that is usable by the pumps 410. In some embodiments the pump may require a fixed voltage, for example a fixed voltage of 32 V, 16V, or 7V. In some embodiments this fixed voltage may be achieved via an ultra-low-power boost converter 502. In some embodiments the boost converter 502 may be designed in order to reduce ferromagnetic content in order to maintain MRI compatibility of the device. In an exemplary embodiment reducing ferromagnetic content may mean using non or minimally ferromagnetic inductors 504. In some embodiments a plurality of ground vias 506 may be used to minimize power consumption.

FIG. 6 may depict an exemplary pump control circuit 600. The exemplary pump control circuit may take the logic inputs from the processor 402 and the power inputs from the voltage control circuit 500 in order to provide a reversible power output to the pump 410. This may include taking the logic output voltage generated by the processor and shifting it to the voltage level of the battery. In some embodiments level shifting may be necessary as the voltage level of the processor may be low in order to conserve power, and therefore may not be high enough on its own to sufficiently switch the transistors of the pump control circuit 600.

FIG. 7 may depict an exemplary h-bridge 700. Once the logic level has been shifted it may be fed into an h-bridge, which may allow for the voltage across the terminals to be high due to the output of the voltage control circuit, and also allow for the voltage to be reversible in order to operate the pump in either direction.

FIG. 8 may depict an exemplary pump management board 800. The pump management board 800 may act as an interface between the hardware and mechanical systems of the pump. In an exemplary embodiment the pump management board may both power and operate sensors on the pump. In an exemplary embodiment the pump management board may be made of, for example, polyamide, gold and/or copper, and may be flexible so as to wrap around the pump and communicate with the implant mainboard, for example by plugging into the implant mainboard via a connector 802. In other embodiments the pump management board may communicate with the implant mainboard through other wired or wireless means, though it may be understood that wireless communication may not be safe and/or viable in some implant devices such as those operating near the brain. In an exemplary embodiment the pump management circuit may provide power to some or all aspects of the pump, for example providing power to one or more electrodes connected to an electroosmotic pump system as described above, which may be done through, for example, one or more power holes 804.

In an exemplary embodiment the pump management board may connect a metallic membrane of the pump to a system ground through one or more ground through holes 806. In some embodiments additional through holes 808 may be used to connect signal lines to the pump electrodes. It may be understood that in an exemplary embodiment the signal lines may work by raising the voltage level of a contact electrode to a system high each time the pump sensing system is enabled. The voltage of the sensing lines may then be read by the system processor 402. In an exemplary embodiment the processor 402 may then be able to detect whether the electrode has initiated contact with the bellows by whether the electrode has gone to system ground.

In some embodiments the pump management board may utilize one or more features in order to prevent or mitigate cracking of traces 810 on the pump management board 800. In some embodiments a stiffener, such as for example a polyamide stiffener, may be placed over one or more of the traces 810, which may help increase the stiffness of the pump management board 800 substrate. In some embodiments the traces may be set so that there are no sharp angles in the traces, for example having round corners 814, which may prevent stress concentrations. In some embodiments the via-trace and pad-trace interfaces may be tapered 816, which may prevent stress concentrations. In some embodiments the traces may be placed so none of the traces are stacked on top of each other 818. In some embodiments multiple or all of the above may be implemented in order to reduce or minimize trace stress.

FIG. 9 may depict an exemplary pump control system logic diagram 900. In a first step 902 a pump enable command may be issued by the processor firmware to the pump control system. In a next step 904 the voltage control circuit may be enabled. In a next step 906 a first logic level and a second level may be set to low, which may initialize the system. In a next step 908 the system may check that there has been sufficient delay to allow for a direction switch, the delay may be necessary to, for example, allow for discharge of system capacitance. In a next step 910 the system may check to see if additional flow rate conditions have been met before pumping can begin. These additional flow rate conditions may include, for example, that an appropriate period of time has passed for the targeted steady flow rate when averaged over a plurality of cycles, that is if the current flow rate is too high or too low the wait period may be modified to correct towards to targeted average. Once flow rate conditions have been met, in a next step 912 the first logic level may be set to high, which may trigger the pump to begin operating in a forward direction. While the pump is pumping in the forward direction, in a next step 914 the pump control system may periodically check whether the forward contact switch has been reached, the contact may be detected, for example, as described above. The frequency that the check is performed may be dependent on the particular application, in some exemplary embodiments the check may be performed every second, in others it may be performed more or less often.

After contact has been detected in step 914, in a next step 916 the pump control system may set both the first logic level and second logic level to low again. In a next step 918 the pump control system may check that there has been sufficient delay to allow for a direction switch, the delay may be necessary to, for example, allow for discharge of system capacitance. In a next step 920 the system may check to see if additional flow rate conditions have been met before pumping can begin in the reverse or backwards direction. Once flow conditions have been met, in a next step 922 the second logic level may be set to high which may begin pumping in the backwards direction. While the pump is pumping in the backward direction, in a next step 924 the pump control system may periodically check whether the forward contact switch has been reached, the contact may be detected, for example, as described above. The frequency that the check is performed may be dependent on the particular application, in some exemplary embodiments the check may be performed every second, in others it may be performed more or less often. Once contact has been detected in step 924 the pump control system may return to step 906.

At any time during the logic sequence described in FIG. 9, a pump disable command may be received.

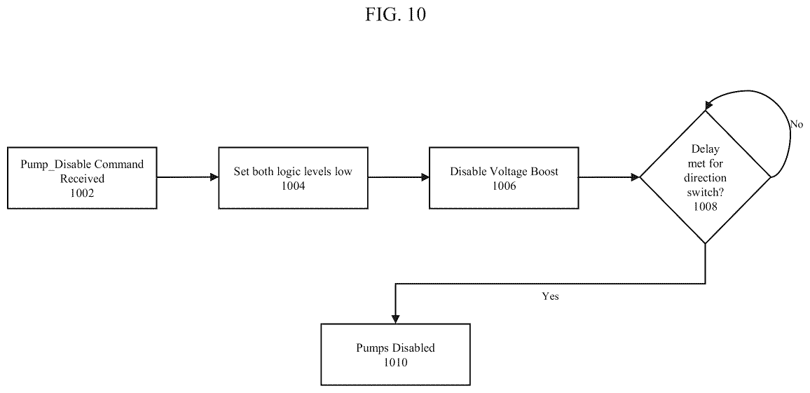

FIG. 10 may depict an exemplary pump control system pump disable command logic diagram 1000. In a first step 1002 the pump control system may receive a Pump_Disable command from the processor. In a second step 1004, the pump control system may set both the first logic level and the second logic level to system low. In a third step 1006 the voltage control circuit may be disabled. In a fourth step 1008 the pump control system may check that there has been sufficient delay to allow for a direction switch, the delay may be necessary to, for example, allow for discharge of system capacitance. In a final step 1010 the pumps may then be disabled.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art. Components Additionally it may be understood that parts or aspects described in one embodiment may likewise be used in other embodiments where appropriate.

Therefore, the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A sensing and control system for an implantable infusion pump comprising:
an implant mainboard comprising a pump control circuit configured to supply a pump voltage;
a pump management board that interfaces with the implant mainboard to receive pump information and deliver the pump voltage;
a battery; and
a voltage control circuit configured to adjust the voltage of the battery.

2. The sensing and control system of claim 1, wherein the pump management board is comprised of polyamide, gold, and/or copper and is flexible.

3. The sensing and control system of claim 1, wherein the voltage control circuit includes at least a low-power boost converter that is MRI safe.

4. The sensing and control system of claim 3, wherein the low-power boost converter is MRI compatible.

5. The sensing and control system of claim 4, wherein the pump voltage is a reversible power output.

6. The sensing and control system of claim 5, wherein the pump voltage is one of +/−7 volts and +/−32 volts.

7. The sensing and control system of claim 4, wherein the pump information includes at least an indication that a pump cycle has been completed; and
wherein the implant mainboard reverses the polarity of the pump voltage when the pump cycle has been completed.

8. The sensing and control system of claim 4, wherein the indication that a pump cycle has completed includes:
an indication that a first conductive membrane has contacted a first electrode; or
an indication that a second conductive membrane has contacted a second electrode; and
wherein when the first conductive membrane has contacted the first electrode or the second membrane has contacted the second electrode, the implant mainboard reverses the polarity of the pump voltage.

9. The sensing and control system of claim 8, wherein after the first conductive membrane has contacted the first electrode or the second membrane has contacted the second electrode and before the polarity of the pump voltage is reserves there is a waiting period.

10. The sensing and control system of claim 2, wherein the pump management board is further comprised of a plurality of traces, wherein each trace is covered by a polyamide stiffener and each of one or more trace corners are rounded corners.

11. An implantable infusion pump with sensing and control system comprising:
an implantable infusion pump configured to deliver a payload fluid;
an implant mainboard, the implant mainboard including a pump control circuit that provides a reversible power output to the implantable infusion pump;
a pump management board which interfaces with the implant mainboard and the infusion pump; and
one or more sensors controlled by the pump management board.

12. The implantable infusion pump with sensing and control system of claim 11, wherein the pump management board is comprised of polyamide, gold and/or copper and is flexible.

13. The implantable infusion pump with sensing and control system of claim 11, further comprising a battery and a voltage control circuit;
the voltage control circuit configured to change the voltage of the battery to a voltage that is usable by the implantable infusion pump.

14. The implantable infusion pump with sensing and control system of claim 13, wherein the voltage control circuit includes at least a low-power boost converter that is MRI safe.

15. The implantable infusion pump with sensing and control system of claim 14, wherein low-power boost converter is MRI compatible.

16. The implantable infusion pump with sensing and control system of claim 11, wherein the implantable infusion pump is further comprised of:
an electroosmotic element;
one or more electrodes which pass through the electroosmotic element;
a first bellows or diaphragm;
a second bellows or diaphragm;
a first contact electrode;
a second contact electrode;
wherein, when the first bellows or diaphragms contacts the first contact electrode or the second bellows or diaphragms contacts the second contact electrode the polarity of the voltage provided by the pump control circuit is reversed.

17. A method for an implantable infusion pump sensing and control system, comprising;
enabling an implantable infusion pump, wherein the implantable infusion pump is configured to dispense a payload fluid;
enabling a voltage control circuit;
continuously checking for a first delay condition;
when the first delay condition is met continuously checking for a first flow rate condition;
when the first flow rate condition is met activating the pump in a forward direction;
detecting a contact switch activation;
continuously checking for a second delay condition;
when the second delay condition is met continuously checking for a second flow rate condition;
when the second flow rate condition is met activating the pump in a backwards direction; and
detecting a second contact switch activation.

18. The method for an implantable infusion pump sensing and control system of claim 17, further comprising continuously alternating the pump between the forwards and backwards directions at least a plurality of times and until a disable command is detected.

19. The method for an implantable infusion pump sensing and control system of claim 18, wherein activating the pump in a forward direction includes sending a first polarity voltage from an implant mainboard to the implantable fusion pump via a pump management board; and
activating the pump in a backwards direction includes sending a second polarity voltage from the implant mainboard to the implantable fusion pump via the pump management board.

20. The method for an implantable infusion pump sensing and control system of claim 19, wherein the pump management board is further comprised of a plurality of traces, wherein each trace is covered by a polyamide stiffener and each of one or more trace corners are rounded corners.
